SEQUENCE LISTING (48 sequences)

SEQ ID NO: 1 - DNA, 25 bp, Artificial Sequence
  Synthetically generated oligonucleotide
acaataatatttgaggcaaatggaa

SEQ ID NO: 2 - DNA, 25 bp, Artificial Sequence
  Synthetically generated oligonucleotide
atgttccttagtcctgtaaccatcc

SEQ ID NO: 3 - DNA, 25 bp, Artificial Sequence
  Synthetically generated oligonucleotide
caataatatttgaggcaaatggaaa

SEQ ID NO: 4 - DNA, 25 bp, Artificial Sequence
  Synthetically generated oligonucleotide
gttccttagtcctgtaaccatcctt

SEQ ID NO: 5 - DNA, 25 bp, Artificial Sequence
  Synthetically generated oligonucleotide
gatgttccttagtcctgtaaccatc

SEQ ID NO: 6 - DNA, 25 bp, Artificial Sequence
  Synthetically generated oligonucleotide
aaatccagaatgtgataggcttcta

SEQ ID NO: 7 - DNA, 25 bp, Artificial Sequence
  Synthetically generated oligonucleotide
agttgttgtatgctgtgtccatcta

SEQ ID NO: 8 - DNA, 25 bp, Artificial Sequence
  Synthetically generated oligonucleotide
tgttgtatgctgtgtccatctatct

SEQ ID NO: 9 - DNA, 25 bp, Artificial Sequence
  Synthetically generated oligonucleotide
ttgttgtatgctgtgtccatctatc

SEQ ID NO: 10 - DNA, 25 bp, Artificial Sequence
  Synthetically generated oligonucleotide
gacaatagtaaaaccgggagacata

SEQ ID NO: 11 - DNA, 25 bp, Artificial Sequence
  Synthetically generated oligonucleotide
gagtgttttgcttaacatatctggg

SEQ ID NO: 12 - DNA, 25 bp, Artificial Sequence
  Synthetically generated oligonucleotide
caaaagaagccaacaaactgtaatc

SEQ ID NO: 13 - DNA, 25 bp, Artificial Sequence
  Synthetically generated oligonucleotide
tatgtctcccggttttactattgtc

SEQ ID NO: 14 - DNA, 25 bp, Artificial Sequence
  Synthetically generated oligonucleotide
agtaaaaccgggagacatacttttg

SEQ ID NO: 15 - DNA, 25 bp, Artificial Sequence
  Synthetically generated oligonucleotide
gagcagaataaaccattttgagaaa

SEQ ID NO: 16 - DNA, 25 bp, Artificial Sequence
  Synthetically generated oligonucleotide
ctttttgataagccataccacattt

SEQ ID NO: 17 - DNA, 25 bp, Artificial Sequence
  Synthetically generated oligonucleotide
ggaatggtcttacatagtggagaag

SEQ ID NO: 18 - DNA, 25 bp, Artificial Sequence
  Synthetically generated oligonucleotide
ttttgataagccataccacatttct

SEQ ID NO: 19 - DNA, 25 bp, Artificial Sequence
  Synthetically generated oligonucleotide
aagagtctgaatgtgcatgtgtaaa

SEQ ID NO: 20 - DNA, 25 bp, Artificial Sequence
  Synthetically generated oligonucleotide
ctccaaattttgattgaaagatacc

SEQ ID NO: 21 - DNA, 24 bp, Artificial Sequence
  Synthetically generated oligonucleotide
ccaaattttgattgaaagataccc

SEQ ID NO: 22 - DNA, 25 bp, Artificial Sequence
  Synthetically generated oligonucleotide
atgtgtaaatggctcttgctttact

SEQ ID NO: 23 - DNA, 25 bp, Artificial Sequence
  Synthetically generated oligonucleotide
caaattttgattgaaagatacccat

SEQ ID NO: 24 - DNA, 25 bp, Artificial Sequence
  Synthetically generated oligonucleotide
acacagtacatgataggacccctta

SEQ ID NO: 25 - DNA, 25 bp, Artificial Sequence
  Synthetically generated oligonucleotide
gtacaagttccattgatacaaacgc

SEQ ID NO: 26 - DNA, 25 bp, Artificial Sequence
  Synthetically generated oligonucleotide
tgtacaagttccattgatacaaacg

SEQ ID NO: 27 - DNA, 24 bp, Artificial Sequence
  Synthetically generated oligonucleotide
agaatacagaaattggtcaaagcc

SEQ ID NO: 28 - DNA, 25 bp, Artificial Sequence
  Synthetically generated oligonucleotide
taaggggtcctatcatgtactgtgt

SEQ ID NO: 29 - DNA, 25 bp, Artificial Sequence
  Synthetically generated oligonucleotide
caacagtactggaaatatgggagtt

SEQ ID NO: 30 - DNA, 25 bp, Artificial Sequence
  Synthetically generated oligonucleotide
agaatcaagcaaaagctgatatgac

SEQ ID NO: 31 - DNA, 25 bp, Artificial Sequence
  Synthetically generated oligonucleotide
attgatatggaatttttcgatggta

SEQ ID NO: 32 - DNA, 25 bp, Artificial Sequence
  Synthetically generated oligonucleotide
aactcccatatttccagtactgttg

SEQ ID NO: 33 - DNA, 25 bp, Caliciviruses
ttaaattctcctcagaaccacattt

SEQ ID NO: 34 - DNA, 24 bp, Caliciviruses
gagaaagaaggtcttctgcgacta

SEQ ID NO: 35 - DNA, 25 bp, Caliciviruses
gagaaagaaggtcttctgcgactac

SEQ ID NO: 36 - DNA, 24 bp, Caliciviruses
agaaagaaggtcttctgcgactac

SEQ ID NO: 37 - DNA, 23 bp, Coronaviruses
tggttttataggtgccacaattc

SEQ ID NO: 38 - DNA, 22 bp, Coronaviruses
gccaattcagtttgtttaccag

SEQ ID NO: 39 - DNA, 25 bp, Coronaviruses
tttataggtgccacaattcgtctac

SEQ ID NO: 40 - DNA, 25 bp, Coronaviruses
ttttataggtgccacaattcgtcta

SEQ ID NO: 41 - DNA, 25 bp, Respiratory syncytial viruses
aaaaacacaacaacaacccaaatac

SEQ ID NO: 42 - DNA, 25 bp, Respiratory syncytial viruses
ttgaacacttcaaagtgaaaatcat

SEQ ID NO: 43 - DNA, 25 bp, Respiratory syncytial viruses
caaagttgaacacttcaaagtgaaa

SEQ ID NO: 44 - DNA, 24 bp, Respiratory syncytial viruses
aaaaacacaacaacaacccaaata

SEQ ID NO: 45 - DNA, 25 bp, Artificial Sequence
  Oligonucleotide
atgtagttcgtgatctaccttctgg

SEQ ID NO: 46 - DNA, 25 bp, Artificial Sequence
  Oligonucleotide
tttaaatagccaacaaaataggctg

SEQ ID NO: 47 - DNA, 25 bp, Artificial Sequence
  Oligonucleotide
aaggctgtaagaatggctctaaaat

SEQ ID NO: 48 - DNA, 25 bp, Artificial Sequence
  Oligonucleotide
gttcgtgatctaccttctggtttta

(19) United States
(12) Patent Application Publication
(10) Pub. No.: US 2010/0105025 A1
(43) Pub. Date: Apr. 29, 2010

(54) DEVICES FOR GENERATING DETECTABLE POLYMERS

(76) Inventor: Eric K. Engelhard, Davis, CA (US)

Correspondence Address: FISH & RICHARDSON P.C., PO BOX 1022, MINNEAPOLIS, MN, 55440-1022, US

(21) Appl. No.: 12/444,120

(22) PCT Filed: Oct. 12, 2007
(86) PCT No.: PCT/US07/81286

§ 371 Date: Apr. 2, 2009

Related U.S. Application Data
(63) Continuation-in-part of application No. 11/548,961, filed on Oct. 12, 2006, now abandoned.
Application No. 11/548,961 is a continuation-in-part of application No. 11/548,980, filed on Oct. 12, 2006, now Pat. No. 7,582,433.
Application No. 11/548,980 is a continuation-in-part of application No. 11/548,989, filed on Oct. 12, 2006, now Pat. No. 7,462,456.
Application No. 11/548,989 is a continuation-in-part of application No. 11/548,997, filed on Oct. 12, 2006, now Pat. No. 7,476,505.
Application No. 11/548,997 is a continuation-in-part of application No. 11/549,008, filed on Oct. 12, 2006, now Pat. No. 7,521,189.
Application No. 11/549,008 is a continuation-in-part of application No. 11/548,986, filed on Oct. 12, 2006.

(51) Int. Cl. C12Q 1/70; C12M 1/34

(52) U.S. Cl. 435/5; 435/287.2

(57) ABSTRACT

This document provides systems, devices, and methods involved in generating detectable polymers. For example, diagnostic systems, diagnostic devices, primer systems, and collections of primer systems are provided.

### CROSS REFERENCE RELATED APPLICATIONS

This document claims priority to U.S. application Ser. Nos. 11/548,961; 11/548,980; 11/548,989; 11/548,997; 11/549,008; and 11/548,986; all filed on Oct. 12, 2006, the contents of which are herein incorporated by reference in their entirety.

### BACKGROUND

1. Technical Field

This document relates to systems, devices, and methods involved in generating detectable polymers.

2. Background Information

Many different types of devices exist for generating polymers such as labeled deoxyribonucleic acids. For example, tubes, tube retainer trays, microtiter plates, microfluidic cards, and glass slides containing arrays have been fabricated to allow a user to generate polymers. The HT7900 Micro Fluidic Card™ is an example of a microfluidic card designed to allow a user to generate polymers. In this case, the microfluidic card functions as a structured array of reaction chambers and contains input ports for inserting samples into the card. The HT7900 Micro Fluidic Card™ is available from Applied Biosystems Group (Foster City, Calif.).

In addition, many different techniques have been developed to detect a generated polymer. For example, machines designed to read fluorescent signals from each well of a microtiter plate have been developed. The FLx800™ reader is an example of an absorbance and fluorescence instrument for measuring samples in various microplate arrangements. The reader can used in numerous fluorescence and absorbance applications in research and routine investigations. Its fluorescence filters are arranged in filter wheels. The reader can handle 6, 48, 96, and 384 well plates and can detect wavelengths in the fluorescence spectral range. Gen5™ data collection and analysis software can be used for data capture, and standard reads and data can be downloaded into Excel for further analysis. Dual optical channels can allow for measurements from above or below the plate. Light to and from the samples can be focused by a lens. The FLx800™ reader is available from BioTek Instruments, Inc. (Winooski, Vt.).

### SUMMARY

This document relates to systems, devices, and methods involved in generating detectable polymers. For example, this document provides diagnostic systems, diagnostic devices, primer systems, and collections of primer systems. A diagnostic system can include a diagnostic device containing a collection of primer systems. This document also provides methods for making diagnostic systems, diagnostic devices, primer systems, and collections of primer systems. For example, this document provides methods for making a diagnostic device containing a collection of primer systems. The systems, devices, and methods provided herein can be used to generate detectable polymers such as amplified deoxyribonucleic acid molecules. In addition, the systems, devices, and methods provided herein can be used to detect influenza A viruses, adenoviruses, caliciviruses, coronaviruses, respiratory syncytial viruses, or SARS coronaviruses within samples. Detecting viruses (e.g., influenza A viruses, adenoviruses, caliciviruses, coronaviruses, respiratory syncytial viruses, or SARS coronaviruses) can help clinicians provide important prognostic information to patients and can help epidemiologists select appropriate viruses for vaccine development.

The description provided herein is based, in part, on the discovery of effective primer systems for generating detectable polymers. For example, a diagnostic device provided herein can contain primer systems effective to detect viruses (e.g., influenza A viruses, adenoviruses, caliciviruses, coronaviruses, respiratory syncytial viruses, or SARS coronaviruses) within samples. Such a diagnostic device can be used to aid clinicians in assessing a patient's prognosis. The description provided herein also is based, in part, on the discovery of primer systems having the ability to not only amplify particular nucleic acid sequences from different viruses (e.g., influenza A viruses, adenoviruses, caliciviruses, coronaviruses, respiratory syncytial viruses, or SARS coronaviruses), but also to not amplify nucleic acid sequences from non-influenza A virus, non-adenovirus, non-calicivirus, non-coronavirus, non-respiratory syncytial virus, or non-SARS coronavirus sources such as a human's genome. In addition, the description provided herein is based, in part, on the discovery of primer systems that can be used simultaneously with a collection of primer pairs under the same amplification reaction conditions to amplify different target nucleic acids if present in the sample being tested. For example, a single diagnostic card having multiple separate microfluidic chambers, each of which contains a different primer system provided herein, can be used in a single amplification reaction to detect the presence or absence of multiple different influenza A viruses. Having the ability to test for the presence or absence of multiple different influenza A viruses using a single diagnostic card with the primer systems provided herein and a single amplification reaction can allow clinicians to detect different influenza A viruses within samples (e.g., a sample collected from a human) accurately and rapidly in a cost effective manner.

In general, one aspect of this document features a device comprising, or consisting essentially of, a housing having a plurality of locations, wherein each of the locations contains an influenza virus primer system, wherein the primers of each influenza virus primer system are between 18 and 28 nucleotides in length and have a theoretical melting temperature between 58° C. and 62° C., wherein the device comprises at least one influenza virus primer system capable of producing an amplification product diagnostic for an H1 target, at least one influenza virus primer system capable of producing an amplification product diagnostic for an H2 target, at least one influenza virus primer system capable of producing an amplification product diagnostic for an H3 target, at least one influenza virus primer system capable of producing an amplification product diagnostic for an H5 target, at least one influenza virus primer system capable of producing an amplification product diagnostic for an N1 target, and at least one influenza virus primer system capable of producing an amplification product diagnostic for an N2 target, and wherein each amplification product, when produced, is between 100 and 400 nucleotides in length. Each of the locations can be a chamber. Each of the locations can be a well. Two or more of the locations can comprise two or more influenza virus primer systems. Each influenza virus primer system of the two or more influenza virus primer systems of a location can be capable of producing an amplification product diagnostic for a different target of influenza A viruses. The primers of each primer system can be between 23 and 27 nucleotides in length. The primers of each influenza virus primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. Each of the locations can comprise an intercalating dye, and each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features a diagnostic device for detecting an influenza virus within a sample. The device comprises, or consists essentially of, a housing having a plurality of locations, wherein each of the locations contains an influenza virus primer system, wherein the primers of each influenza virus primer system are between 18 and 28 nucleotides in length and have a theoretical melting temperature between 58° C. and 62° C., wherein the device is capable of producing an amplification product diagnostic for each of at least six different targets of influenza A viruses, and wherein each amplification product, when produced, is between 100 and 400 nucleotides in length. Each of the locations can be a chamber. Each of the locations can be a well. Two or more of the locations can comprise two or more influenza virus primer systems. Each influenza virus primer system of the two or more influenza virus primer systems of a location can be capable of producing an amplification product diagnostic for a different target of influenza A viruses. The primers of each influenza virus primer system can be between 23 and 27 nucleotides in length. The primers of each influenza virus primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. The at least six different targets of influenza A viruses can be H1, H2, H3, H5, N1, and N2 targets. Each of the locations can comprise an intercalating dye, and each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features a method for detecting an influenza A virus within a sample. The method comprises, or consists essentially of, (a) performing a nucleic acid amplification reaction using the sample as a source of template and a diagnostic device, wherein the device comprises, or consists essentially of, a housing having a plurality of locations, wherein each of the locations contains an influenza virus primer system, wherein the primers of each influenza virus primer system are between 18 and 28 nucleotides in length and have a theoretical melting temperature between 58° C. and 62° C., wherein the device is capable of producing an amplification product diagnostic for at least six different targets of influenza A viruses, and wherein each amplification product, when produced, is between 100 and 400 nucleotides in length, and (b) determining which locations of the device contain an influenza virus primer system that resulted in the formation of amplification product, thereby detecting an influenza A virus. The sample can be a sample obtained from a human. The nucleic acid amplification reaction can comprise at least 10 cycles. The nucleic acid amplification reaction can comprise at least 20 cycles. The nucleic acid amplification reaction can comprise a denaturing step at about 94° C. or about 95° C. The nucleic acid amplification reaction can comprise an annealing step at about 60° C. The nucleic acid amplification reaction can comprise an extension step at about 72° C. The sample can be a mucus sample. The sample can be a sample obtained from the human using a swab. The sample can be a sample processed to obtain viral nucleic acid. The amplification reaction can be performed in a thermal cycler device configured to receive the diagnostic device. The determining step (b) can be performed in using a dye reader device configured to receive the diagnostic device. Each of the locations can comprise an intercalating dye, wherein each amplification product, when produced, is labeled with the intercalating dye, and wherein determining which locations of the device contain a primer system that resulted in the formation of amplification product is based on a signal from the dye. The amplification reaction and the determining step (b) can be performed in a machine configured to receive the diagnostic device, the machine comprising a thermal cycler device and a dye reader device. The machine can be capable of providing output indicating the presence of the influenza A virus. The machine can be capable of providing output indicating the influenza virus primer systems that detected the presence of the influenza A virus. The output can be paper printout or a computer readable file.

In another aspect, this document features a device comprising, or consisting essentially of, a housing having a plurality of locations, wherein each of the locations contains an adenovirus primer system, wherein the primers of each adenovirus primer system are between 18 and 28 nucleotides in length and have a theoretical melting temperature between 58° C. and 62° C., wherein the device comprises at least one adenovirus primer system capable of producing an amplification product diagnostic for an adenovirus, and wherein each amplification product, when produced, is between 100 and 400 nucleotides in length. Each of the locations can be a chamber. Each of the locations can be a well. The primers of each adenovirus primer system can be between 23 and 27 nucleotides in length. The primers of each adenovirus primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. Each of the locations can comprise an intercalating dye, and wherein each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features method for detecting an adenovirus within a sample. The method comprises, or consists essentially of, (a) performing a nucleic acid amplification reaction using the sample as a source of template and a diagnostic device, wherein the device comprises a housing having a plurality of locations, wherein each of the locations contains an adenovirus primer system, wherein the primers of each adenovirus primer system are between 18 and 28 nucleotides in length and have a theoretical melting temperature between 58° C. and 62° C., wherein the device is capable of producing an amplification product diagnostic for an adenovirus, and wherein each amplification product, when produced, is between 100 and 400 nucleotides in length, and (b) determining which locations of the device contain an adenovirus primer system that resulted in the formation of amplification product, thereby detecting an adenovirus. The sample can be a sample obtained from a human. The nucleic acid amplification reaction can comprise at least 10 cycles. The nucleic acid amplification reaction can comprise at least 20 cycles. The nucleic acid amplification reaction can comprise a denaturing step at about 94° C. or about 95° C. The nucleic acid amplification reaction can comprise an annealing step at about 60° C. The nucleic acid amplification reaction can comprise an extension step at about 72° C. The sample can be a mucus sample. The sample can be a sample obtained from the human using a swab. The sample can be a sample processed to obtain viral nucleic acid. Each of the locations can comprise an intercalating dye, wherein each amplification product, when produced, is labeled with the intercalating dye, and wherein determining which locations of the device contain an adenovirus primer system that resulted in the formation of amplification product is based on a signal from the dye. The amplification reaction can be performed in a thermal cycler device configured to receive the diagnostic device. The determining step (b) can be performed in using a dye reader device configured to receive the diagnostic device. The amplification reaction and the determining step (b) can be performed in a machine configured to receive the diagnostic device, the machine comprising a thermal cycler device and a dye reader device. The machine can be capable of providing output indicating the presence of the adenovirus. The machine can be capable of providing output indicating the adenovirus primer system that detected the presence of the adenovirus. The output can be a paper printout or a computer readable file.

In another aspect, this document features a device comprising, or consisting essentially of, a housing having a plurality of locations, wherein each of the locations contains a calicivirus primer system, wherein the primers of each calicivirus primer system are between 18 and 28 nucleotides in length and have a theoretical melting temperature between 58° C. and 62° C., wherein the device comprises at least one calicivirus primer system capable of producing an amplification product diagnostic for an calicivirus, and wherein each amplification product, when produced, is between 100 and 400 nucleotides in length. Each of the locations can be a chamber. Each of the locations can be a well. The primers of each calicivirus primer system can be between 23 and 27 nucleotides in length. The primers of each calicivirus primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. Each of the locations can comprise an intercalating dye, and wherein each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features method for detecting a calicivirus within a sample. The method comprises, or consists essentially of, (a) performing a nucleic acid amplification reaction using the sample as a source of template and a diagnostic device, wherein the device comprises a housing having a plurality of locations, wherein each of the locations contains a calicivirus primer system, wherein the primers of each calicivirus primer system are between 18 and 28 nucleotides in length and have a theoretical melting temperature between 58° C. and 62° C., wherein the device is capable of producing an amplification product diagnostic for a calicivirus, and wherein each amplification product, when produced, is between 100 and 400 nucleotides in length, and (b) determining which locations of the device contain a calicivirus primer system that resulted in the formation of amplification product, thereby detecting a calicivirus. The sample can be a sample obtained from a human. The nucleic acid amplification reaction can comprise at least 10 cycles. The nucleic acid amplification reaction can comprise at least 20 cycles. The nucleic acid amplification reaction can comprise a denaturing step at about 94° C. or about 95° C. The nucleic acid amplification reaction can comprise an annealing step at about 60° C. The nucleic acid amplification reaction can comprise an extension step at about 72° C. The sample can be a mucus sample. The sample can be a sample obtained from the human using a swab. The sample can be a sample processed to obtain viral nucleic acid. Each of the locations can comprise an intercalating dye, wherein each amplification product, when produced, is labeled with the intercalating dye, and wherein determining which locations of the device contain a primer system that resulted in the formation of amplification product is based on a signal from the dye. The amplification reaction can be performed in a thermal cycler device configured to receive the diagnostic device. The determining step (b) can be performed in using a dye reader device configured to receive the diagnostic device. The amplification reaction and the determining step (b) can be performed in a machine configured to receive the diagnostic device, the machine comprising a thermal cycler device and a dye reader device. The machine can be capable of providing output indicating the presence of the calicivirus. The machine can be capable of providing output indicating the calicivirus primer system that detected the presence of the calicivirus. The output can be a paper printout or a computer readable file.

In another aspect, this document features a device comprising, or consisting essentially of, a housing having a plurality of locations, wherein each of the locations contains a coronavirus primer system, wherein the primers of each coronavirus primer system are between 18 and 28 nucleotides in length and have a theoretical melting temperature between 58° C. and 62° C., wherein the device comprises at least one primer system capable of producing an amplification product diagnostic for a coronavirus, and wherein each amplification product, when produced, is between 100 and 400 nucleotides in length. Each of the locations can be a chamber. Each of the locations can be a well. The primers of each coronavirus primer system can be between 23 and 27 nucleotides in length. The primers of each coronavirus primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. Each of the locations can comprise an intercalating dye, and wherein each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features method for detecting a coronavirus within a sample. The method comprises, or consists essentially of, (a) performing a nucleic acid amplification reaction using the sample as a source of template and a diagnostic device, wherein the device comprises a housing having a plurality of locations, wherein each of the locations contains a coronavirus primer system, wherein the primers of each coronavirus primer system are between 18 and 28 nucleotides in length and have a theoretical melting temperature between 58° C. and 62° C., wherein the device is capable of producing an amplification product diagnostic for a coronavirus, and wherein each amplification product, when produced, is between 100 and 400 nucleotides in length, and (b) determining which locations of the device contain a primer system that resulted in the formation of amplification product, thereby detecting a coronavirus. The sample can be a sample obtained from a human. The nucleic acid amplification reaction can comprise at least 10 cycles. The nucleic acid amplification reaction can comprise at least 20 cycles. The nucleic acid amplification reaction can comprise a denaturing step at about 94° C. or about 95° C. The nucleic acid amplification reaction can comprise an annealing step at about 60° C. The nucleic acid amplification reaction can comprise an extension step at about 72° C. The sample can be a mucus sample. The sample can be a sample obtained from the human using a swab. The sample can be a sample processed to obtain viral nucleic acid. Each of the locations can comprise an intercalating dye, wherein each amplification product, when produced, is labeled with the intercalating dye, and wherein determining which locations of the device contain a primer system that resulted in the formation of amplification product is based on a signal from the dye. The amplification reaction can be performed in a thermal cycler device configured to receive the diagnostic device. The determining step (b) can be performed in using a dye reader device configured to receive the diagnostic device. The amplification reaction and the determining step (b) can be performed in a machine configured to receive the diagnostic device, the machine comprising a thermal cycler device and a dye reader device. The machine can be capable of providing output indicating the presence of the coronavirus. The machine can be capable of providing output indicating the coronavirus primer system that detected the presence of the coronavirus. The output can be a paper printout or a computer readable file.

In another aspect, this document features a device comprising, or consisting essentially of, a housing having a plurality of locations, wherein each of the locations contains a respiratory syncytial virus primer system, wherein the primers of each respiratory syncytial virus primer system are between 18 and 28 nucleotides in length and have a theoretical melting temperature between 58° C. and 62° C., wherein the device comprises at least one respiratory syncytial virus primer system capable of producing an amplification product diagnostic for a respiratory syncytial virus, and wherein each amplification product, when produced, is between 100 and 400 nucleotides in length. Each of the locations can be a chamber. Each of the locations can be a well. The primers of each respiratory syncytial virus primer system can be between 23 and 27 nucleotides in length. The primers of each respiratory syncytial virus primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. Each of the locations can comprise an intercalating dye, and wherein each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features method for detecting a respiratory syncytial virus within a sample. The method comprises, or consists essentially of, (a) performing a nucleic acid amplification reaction using the sample as a source of template and a diagnostic device, wherein the device comprises a housing having a plurality of locations, wherein each of the locations contains a respiratory syncytial virus primer system, wherein the primers of each respiratory syncytial virus primer system are between 18 and 28 nucleotides in length and have a theoretical melting temperature between 58° C. and 62° C., wherein the device is capable of producing an amplification product diagnostic for a respiratory syncytial virus, and wherein each amplification product, when produced, is between 100 and 400 nucleotides in length, and (b) determining which locations of the device contain a primer system that resulted in the formation of amplification product, thereby detecting a respiratory syncytial virus. The sample can be a sample obtained from a human. The nucleic acid amplification reaction can comprise at least 10 cycles. The nucleic acid amplification reaction can comprise at least 20 cycles. The nucleic acid amplification reaction can comprise a denaturing step at about 94° C. or about 95° C. The nucleic acid amplification reaction can comprise an annealing step at about 60° C. The nucleic acid amplification reaction can comprise an extension step at about 72° C. The sample can be a mucus sample. The sample can be a sample obtained from the human using a swab. The sample can be a sample processed to obtain viral nucleic acid. Each of the locations can comprise an intercalating dye, wherein each amplification product, when produced, is labeled with the intercalating dye, and wherein determining which locations of the device contain a primer system that resulted in the formation of amplification product is based on a signal from the dye. The amplification reaction can be performed in a thermal cycler device configured to receive the diagnostic device. The determining step (b) can be performed in using a dye reader device configured to receive the diagnostic device. The amplification reaction and the determining step (b) can be performed in a machine configured to receive the diagnostic device, the machine comprising a thermal cycler device and a dye reader device. The machine can be capable of providing output indicating the presence of the respiratory syncytial virus. The machine can be capable of providing output indicating the respiratory syncytial virus primer system that detected the presence of the respiratory syncytial virus. The output can be a paper printout or a computer readable file.

In another aspect, this document features a device comprising, or consisting essentially of, a housing having a plurality of locations, wherein each of the locations contains a SARS coronavirus primer system, wherein the primers of each SARS coronavirus primer system are between 18 and 28 nucleotides in length and have a theoretical melting temperature between 58° C. and 62° C., wherein the device comprises at least one SARS coronavirus primer system capable of producing an amplification product diagnostic for a SARS coronavirus, and wherein each amplification product, when produced, is between 100 and 400 nucleotides in length. Each of the locations can be a chamber. Each of the locations can be a well. The primers of each SARS coronavirus primer system can be between 23 and 27 nucleotides in length. The primers of each SARS coronavirus primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. Each of the locations can comprise an intercalating dye, and wherein each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features method for detecting a SARS coronavirus within a sample. The method comprises, or consists essentially of, (a) performing a nucleic acid amplification reaction using the sample as a source of template and a diagnostic device, wherein the device comprises a housing having a plurality of locations, wherein each of the locations contains a SARS coronavirus primer system, wherein the primers of each SARS coronavirus primer system are between 18 and 28 nucleotides in length and have a theoretical melting temperature between 58° C. and 62° C., wherein the device is capable of producing an amplification product diagnostic for a SARS coronavirus, and wherein each amplification product, when produced, is between 100 and 400 nucleotides in length, and (b) determining which locations of the device contain a primer system that resulted in the formation of amplification product, thereby detecting a SARS coronavirus. The sample can be a sample obtained from a human. The nucleic acid amplification reaction can comprise at least 10 cycles. The nucleic acid amplification reaction can comprise at least 20 cycles. The nucleic acid amplification reaction can comprise a denaturing step at about 94° C. or about 95° C. The nucleic acid amplification reaction can comprise an annealing step at about 60° C. The nucleic acid amplification reaction can comprise an extension step at about 72° C. The sample can be a mucus sample. The sample can be a sample obtained from the human using a swab. The sample can be a sample processed to obtain viral nucleic acid. Each of the locations can comprise an intercalating dye, wherein each amplification product, when produced, is labeled with the intercalating dye, and wherein determining which locations of the device contain a primer system that resulted in the formation of amplification product is based on a signal from the dye. The amplification reaction can be performed in a thermal cycler device configured to receive the diagnostic device. The determining step (b) can be performed in using a dye reader device configured to receive the diagnostic device. The amplification reaction and the determining step (b) can be performed in a machine configured to receive the diagnostic device, the machine comprising a thermal cycler device and a dye reader device. The machine can be capable of providing output indicating the presence of the SARS coronavirus. The machine can be capable of providing output indicating the SARS coronavirus primer system that detected the presence of the SARS coronavirus. The output can be a paper printout or a computer readable file.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a top view of a microfluidic card.

### DETAILED DESCRIPTION

This document provides systems, devices, and methods involved in generating detectable polymers. For example, this document provides diagnostic systems, diagnostic devices, primer systems, and collections of primer systems. A diagnostic system can include a diagnostic device containing primer systems.

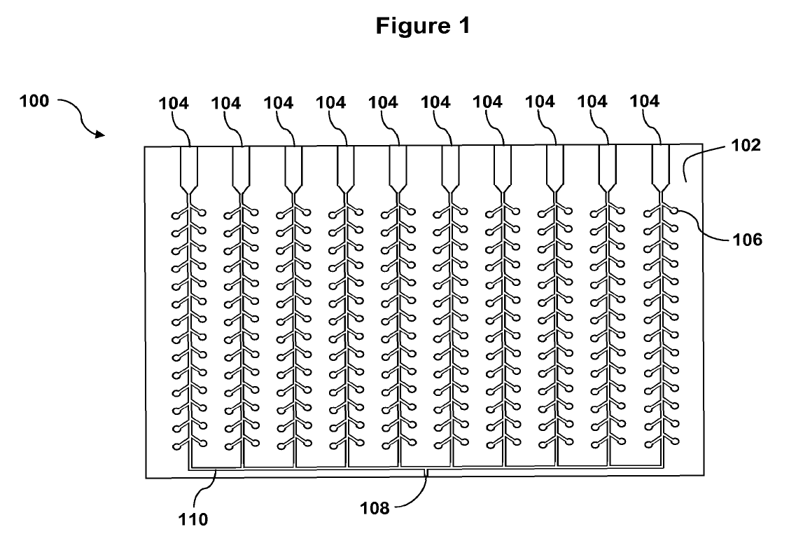

In general, a diagnostic device provided herein can include a housing having a plurality of locations. The housing can be any shape and size and can be made from any type of material including, without limitation, plastic, glass, silicone, or metal. For example, a housing provided herein can be rectangular, square, circular, or oval in shape, and can have a length, width, or diameter between five cm and 50 cm (e.g., between ten cm and 40 cm, between ten cm and 30 cm, or between ten cm and 25 cm). The depth or height of a housing provided herein can be between 0.2 cm and 2 cm (e.g., between 0.2 and 1 cm, between 0.3 and 1 cm, or between 0.5 and 1 cm). Each location of a housing can be configured to allow an amplification reaction to occur without primer system contamination from other locations. The locations of a housing provided herein can be any shape or size. For example, the locations of a housing provided herein can be in the configuration of a well or chamber with, for example, the ability to hold a volume between 1 μL and 100 μL (e.g., between 1μL and 20 μL, between 1 μL and 10 μL, between 1 μL and 5 μL, between 10 μL and 50 μL, or between 15 μL and 25 μL). Such a volume can be 1.5 μL, 10 μL, 20 μL, or 30 μL. In some cases, a housing can be a 96-well plate with each location being a well of the 96-well plate. A diagnostic device can be in the form of a microfluidic card. Such a card can have a series of locations and channels. The channels can provide fluid communication between a sample inlet port and one or more locations. For example, a housing can be a mircofluidic card having one or more sample inlet ports in fluid communication with one or more locations via one or more channels. In some cases, such a housing can include one or more outlet ports for providing an outlet for added solutions or for providing an outlet for air so that fluid can flow through the channels. In one embodiment, a diagnostic device provided herein can be in the form of a microfluidic card with eight sample inlet ports each connected through channels (e.g., microcapillaries) to 48 locations (e.g., reaction chambers). Another example of a microfluidic card design is depicted in FIG. 1.

With reference to FIG. 1, microfluidic card 100 can have housing 102 defining a plurality of locations 106. While 280 separate locations are shown in this example, a housing provided herein can define any number of locations (e.g., 10, 25, 48, 96, 384, 1536, or more locations). Each location 106 can be in fluid communication with a sample inlet port 104 and an outlet port 108 via channel 110. Any number of channels can be defined by housing 102. For example, a housing provided herein can define one continuous, interconnected channel or can contain multiple separate channels.

A diagnostic device provided herein can contain a collection of primer systems and primer pairs. For example, each primer system or primer pair of a collection can be located at a different location defined by a housing so as to isolate each primer system or primer pair from the other primer systems or primer pairs of the collection. For example, each primer system or primer pair of a collection can be housed within a separate location (e.g., a separate well of a plastic microtiter plate or a separate chamber of a microfluidic card). In some cases, each primer system or primer pair of a collection or a subset of primer systems or primer pairs of a collection can be housed together. For example, one primer system provided herein and one primer pair of a collection of 50 primer systems and primer pairs can be housed within a single well of a plastic microtiter plate with the remaining 48 primer systems and primer pairs being housed within separate wells.

In some cases, a system or diagnostic device provided herein can contain at least six different primer systems set forth in Table 1 (e.g., at least seven, at least eight, at least nine, at least ten, at least eleven, at least 12, at least 13, at least 14, at least 15, at least 16, at least 17, or at least 18 different primer systems set forth in Table 1). In addition to containing any one or more of the primer systems set forth in Table 1 in any combination, a diagnostic device can contain primer systems not listed in Table 1. For example, a diagnostic device can contain a primer system similar to primer system number 1 with the exception that each nucleic acid primer is two nucleotides shorter than those of primer system number 1. In some cases, a diagnostic device can contain a primer pair designed to amplify host nucleic acid (e.g., human genomic nucleic acid or mRNA).

TABLE 1
Optimal influenza virus primer systems for each target.

| Primer | | SEQ | | | | |
|---|---|---|---|---|---|---|
| System No. | Primer Sequence | ID NO: | Length | Tm | Target | Hits* |
| 1 | ACAATAATATTTGAGGCAAATGGAA | 1 | 25 | 60.0 | H1 | 457 |
| | ATGTTCCTTAGTCCTGTAACCATCC | 2 | 25 | 60.8 | | |
| 2 | CAATAATATTTGAGGCAAATGGAAA | 3 | 25 | 60.4 | H1 | 450 |
| | GTTCCTTAGTCCTGTAACCATCCTT | 4 | 25 | 60.2 | | |
| 3 | ACAATAATATTTGAGGCAAATGGAA | 1 | 25 | 60.0 | H1 | 450 |
| | GATGTTCCTTAGTCCTGTAACCATC | 5 | 25 | 59.4 | | |
| 4 | AAATCCAGAATGTGATAGGCTTCTA | 6 | 25 | 59.6 | H2 | 62 |
| | AGTTGTTGTATGCTGTGTCCATCTA | 7 | 25 | 60.0 | | |
| 5 | AAATCCAGAATGTGATAGGCTTCTA | 6 | 25 | 59.6 | H2 | 59 |
| | TGTTGTATGCTGTGTCCATCTATCT | 8 | 25 | 60.0 | | |
| 6 | AAATCCAGAATGTGATAGGCTTCTA | 6 | 25 | S9.6 | H2 | 59 |
| | TTGTTGTATGCTGTGTCCATCTATC | 9 | 25 | 60.3 | | |
| 7 | GACAATAGTAAAACCGGGAGACATA | 10 | 25 | S9.7 | H3 | 1842 |
| | GAGTGTTTTGCTTAACATATCTGGG | 11 | 25 | 60.3 | | |
| 8 | CAAAAGAAGCCAACAAACTGTAATC | 12 | 2S | 60.4 | H3 | 1834 |
| | TATGTCTCCCGGTTTTACTATTGTC | 13 | 2S | S9.7 | | |
| 9 | AGTAAAACCGGGAGACATACTTTTG | 14 | 2S | 60.9 | H3 | 1726 |
| | GAGTGTTTTGCTTAACATATCTGGG | 11 | 25 | 60.3 | | |
| 10 | GAGCAGAATAAACCATTTTGAGAAA | 15 | 25 | 60.0 | H5 | 497 |
| | CTTTTTGATAAGCCATACCACATTT | 16 | 25 | 59.7 | | |
| 11 | GGAATGGTCTTACATAGTGGAGAAG | 17 | 25 | 59.5 | H5 | 488 |
| | TTTTGATAAGCCATACCACATTTCT | 18 | 25 | 60.1 | | |
| 12 | GGAATGGTCTTACATAGTGGAGAAG | 17 | 25 | 59.5 | H5 | 488 |
| | CTTTTTGATAAGCCATACCACATTT | 16 | 25 | 59.7 | | |
| 13 | AAGAGTCTGAATGTGCATGTGTAAA | 19 | 25 | 60.1 | N1 | 430 |
| | CTCCAAATTTTGATTGAAAGATACC | 20 | 25 | 59.3 | | |
| 14 | AAGAGTCTGAATGTGCATGTGTAAA | 19 | 25 | 60.1 | N1 | 429 |
| | CCAAATTTTGATTGAAAGATACCC | 21 | 24 | 60.0 | | |
| 15 | ATGTGTAAATGGCTCTTGCTTTACT | 22 | 25 | 59.7 | N1 | 427 |
| | CAAATTTTGATTGAAAGATACCCAT | 23 | 25 | 59.6 | | |
| 16 | ACACAGTACATGATAGGACCCCTTA | 24 | 25 | 60.1 | N2 | 1050 |
| | GTACAAGTTCCATTGATACAAACGC | 25 | 25 | 61.0 | | |
| 17 | ACACAGTACATGATAGGACCCCTTA | 24 | 25 | 60.1 | N2 | 1044 |
| | TGTACAAGTTCCATTGATACAAACG | 26 | 25 | 60.2 | | |
| 18 | AGAATACAGAAATTGGTCAAAGCC | 27 | 24 | 59.9 | N2 | 1027 |
| | TAAGGGGTCCTATCATGTACTGTGT | 28 | 25 | 60.1 | | |

*total number of different gi numbers for the indicated target that is available in GenBank with nucleic acid sequences aligning with each primer of the indicated primer system.

The term “influenza virus primer system” as used herein refers to a combination of two nucleic acid primers having the ability to amplify nucleic acid provided that the sequence of each nucleic acid primer is from 15 to 50 nucleotides in length and is such that it aligns without a mismatch to a sequence, or its complement, set forth in a GenBank gi number listed in Table 2. For example, each primer of an influenza virus primer system provided herein can be from 15 to 45 nucleotides the length. In some cases, each primer of an influenza virus primer system provided herein can range from 20 to 40 nucleotides (e.g., from 20 to 35 nucleotides, from 20 to 30 nucleotides, or from 21 to 28 nucleotides). The influenza virus primer systems provided herein can be selected such that the length of amplified target nucleic acid, if present within an amplification reaction, would be between 100 and 400 nucleotides (e.g., between 150 and 350 nucleotides, between 175 and 325 nucleotides, or between 200 and 300 nucleotides). The theoretical melting temperature of each primer of an influenza virus primer system provided herein can be between 58° C. and 62° C. (e.g., between 59° C. and 61° C.). A primer's theoretical melting temperature is calculated as follows:

Tm=81.5+16.6(log 10([Na+]))+0.41*(% GC)−600/length

where [Na+] is 0.005 M. Each influenza virus primer system provided herein can be used to amplify nucleic acid present in an influenza virus (e.g., influenza A virus).

TABLE 2
Representative gi numbers for each influenza virus primer system.

| Primer | |
|---|---|
| System No. | gi number |
| 1 | 14587062; 14587044; 324121; 57471016; 89779320; |
| | 3831768; 324166; 89903074; 89152214; 14587071; |
| | 21717601; 21717603; 21717605; 20068260; 60776; |
| | and 14587064 |
| 2 | 14587062; 221341; 57471016; 3831768; 14587071; |
| | 20068260; 60776; and 14587064 |
| 3 | 14587062; 14587048; 324121; 57471016; 89903074; |
| | 89152214; 14587071; 21717601; 21717603; 21717605; |
| | 60776; and 14587064 |
| 4, 5, 6 | 37784911; 13182908; and 221323 |
| 7 | 38154801; 75756549; 39980625; 4760441; 9988667; |
| | 62125815; and 71558924 |
| 8 | 38154801; 75756549; 52082719; 4760441; 9988667; |
| | 58198684; 73914160; 79082902; 71558924; 70673281; |
| | and 1236532 |
| 9 | 38154801; 6177877; 39980625; 9988667; 62125817; |
| | 4760445; 71558924; and 5764316 |
| 10 | 47716772; 46244245; 4240441; 57924521; 66473402; |
| | 51628336; 46361439; 78146041; 57924375; 93008346; |
| | and 46361453 |
| 11, 12 | 47716772; 46244245; 11596241; 4240441; 12619372; |
| | 66473402; 93008346; and 46361453 |
| 13, 14 | 46360356; 81171258; 5805284; 47834869; 28849520; |
| | 45934600; 71277732; 56673057; 50296188; 47834873; |
| | 88687197; and 41057636 |
| 15 | 46360356; 81171258; 5805284; 47834869; 28849520; |
| | 45934600; 71277732; 56673057; 50296188; 47834873; |
| | and 88687197 |
| 16, 17 | 59896304; 17223938; 22859388; 56159974; 73665372; |
| | 66947418; and 22859374 |
| 18 | 59896304; 56159974; 73665372; 66947418; and 66947411 |

The influenza virus primer systems provided herein can share unifying advantageous features. For example, each influenza virus primer system provided herein can amplify nucleic acid from influenza A viruses. In addition, influenza virus primer systems provided herein can be selected such that the length of amplified viral nucleic acid would be between 100 and 400 nucleotides. Moreover, the theoretical melting temperature of the influenza virus primer systems provided herein can be uniformly between 58° C. and 62° C., and the length of each primer of the influenza virus primer systems provided herein can range from 15 to 50 nucleotides (e.g., from 21 to 28 nucleotides). These unifying characteristics can contribute to the effective detection of nucleic acid from influenza A viruses present within samples.

The first primer system listed in Table 1 for each of the different targets (e.g., H1, H2, H3, H5, N1, and N2) can be used to make a collection of at least six different primer systems and can be used effectively to detect a large group of different influenza A viruses. For example, primer system number 1 can have the ability to detect H1 nucleic acid sequences associated with 457 different GenBank gi numbers. Primer system number 4 can have the ability to detect H2 nucleic acid sequences associated with 62 different GenBank gi numbers. Primer system number 7 can have the ability to detect H3 nucleic acid sequences associated with 1842 different GenBank gi numbers. Primer system number 10 can have the ability to detect H5 nucleic acid sequences associated with 497 different GenBank gi numbers. Primer system number 13 can have the ability to detect N1 nucleic acid sequences associated with 430 different GenBank gi numbers. Primer system number 16 can have the ability to detect N2 nucleic acid sequences associated with 1050 different GenBank gi numbers.

In some cases, a system or diagnostic device provided herein can contain at least one primer system set forth in Table 3 (e.g., at least two primer systems set forth in Table 3). In addition to containing any one or more of the primer systems set forth in Table 3 in any combination, a diagnostic device can contain primer systems not listed in Table 3. For example, a diagnostic device can contain a primer system similar to primer system number 19 with the exception that each nucleic acid primer is two nucleotides shorter than those of primer system number 19. In some cases, a diagnostic device can contain a primer pair designed to amplify host nucleic acid (e.g., human genomic nucleic acid or mRNA).

TABLE 3
Optimal adenovirus primer systems for adenoviruses.

| Primer | | | | | |
|---|---|---|---|---|---|
| System No. | Primer Sequence | SEQ ID NO: | Length | Tm | Hits* |
| 19 | CAACAGTACTGGAAATATGGGAGTT | 29 | 25 | 59.7 | 89 |
| | AGAATCAAGCAAAAGCTGATATGAC | 30 | 25 | 60.2 | |
| 20 | ATTGATATGGAATTTTTCGATGGTA | 31 | 25 | 59.8 | 85 |
| | AACTCCCATATTTCCAGTACTGTTG | 32 | 25 | 59.7 | |
| 21 | TATTGATATGGAATTTTTCGATGGT | 33 | 25 | 59.8 | 85 |
| | AACTCCCATATTTCCAGTACTGTTG | 32 | 25 | 59.7 | |

*total number of different gi numbers that is available in GenBank with nucleic acid sequences aligning with each primer of the indicated primer system.

The term “adenovirus primer system” as used herein refers to a combination of two nucleic acid primers having the ability to amplify nucleic acid provided that the sequence of each nucleic acid primer is from 15 to 50 nucleotides in length and is such that it aligns without a mismatch to a sequence, or its complement, set forth in a GenBank gi number listed in Table 4. For example, each primer of an adenovirus primer system provided herein can be from 15 to 45 nucleotides the length. In some cases, each primer of an adenovirus primer system provided herein can range from 20 to 40 nucleotides (e.g., from 20 to 35 nucleotides, from 20 to 30 nucleotides, or from 21 to 28 nucleotides). The adenovirus primer systems provided herein can be selected such that the length of amplified target nucleic acid, if present within an amplification reaction, would be between 100 and 400 nucleotides (e.g., between 150 and 350 nucleotides, between 175 and 325 nucleotides, or between 200 and 300 nucleotides). The theoretical melting temperature of each primer of an adenovirus primer system provided herein can be between 58° C. and 62° C. (e.g., between 59° C. and 61° C.). Each adenovirus primer system provided herein can be used to amplify nucleic acid present in an adenovirus.

TABLE 4
Representative gi numbers for adenovirus primer systems.

| Primer | |
|---|---|
| System No. | gi number |
| 19 | 3641840; 15076670; 1710306; 3582745; 37702194; |
| | 11641372; 93302091; 11968173; 57115749; 83017238; |
| | 40786870; 51173294; 55847711; 57115983; 16930159; |
| | 37702204; 434911; 74229858; and 2511449 |
| 20, 21 | 3641840; 15076670; 1710308; 3582745; 37702194; |
| | 11641372; 93302091; 57115749; 83017238; 40786870; |
| | 55847711; 57115983; 16930159; 434911; 74229858; |
| | and 69139752 |

The adenovirus primer systems provided herein can share unifying advantageous features. For example, each adenovirus primer system provided herein can amplify nucleic acid from adenoviruses. In addition, adenovirus primer systems provided herein can be selected such that the length of amplified viral nucleic acid would be between 100 and 400 nucleotides. Moreover, the theoretical melting temperature of the adenovirus primer systems provided herein can be uniformly between 58° C. and 62° C., and the length of each primer of the adenovirus primer systems provided herein can range from 15 to 50 nucleotides (e.g., from 21 to 28 nucleotides). These unifying characteristics can contribute to the effective detection of nucleic acid from adenoviruses present within samples.

The adenovirus primer systems listed in Table 3 can be used effectively to detect a large group of different adenoviruses. For example, adenovirus primer system number 19 can have the ability to detect adenovirus nucleic acid sequences associated with 89 different GenBank gi numbers.

In some cases, a system or diagnostic device provided herein can contain at least one primer system set forth in Table 5 (e.g., at least two primer systems set forth in Table 5). In addition to containing any one or more of the primer systems set forth in Table 5 in any combination, a diagnostic device can contain primer systems not listed in Table 5. For example, a diagnostic device can contain a primer system similar to primer system number 22 with the exception that each nucleic acid primer is two nucleotides shorter than those of primer system number 22. In some cases, a diagnostic device can contain a primer pair designed to amplify host nucleic acid (e.g., human genomic nucleic acid or mRNA).

TABLE 5
Optimal calicivirus primer systems for caliciviruses.

| Primer | | | | | |
|---|---|---|---|---|---|
| System No. | Primer Sequence | SEQ ID NO: | Length | Tm | Hits* |
| 22 | TTAAATTCTCCTCAGAACCACATTT | 33 | 25 | 59.5 | 36 |
| | GAGAAAGAAGGTCTTCTGCGACTA | 34 | 24 | 60.5 | |
| 23 | TTAAATTCTCCTCAGAACCACATTT | 33 | 25 | 59.5 | 35 |
| | GAGAAAGAAGGTCTTCTGCGACTAC | 35 | 25 | 61.2 | |
| 24 | TTAAATTCTCCTCAGAACCACATTT | 33 | 25 | 59.5 | 35 |
| | AGAAAGAAGGTCTTCTGCGACTAC | 36 | 24 | 59.6 | |

*total number of different gi numbers that is available in GenBank with nucleic acid sequences aligning with each primer of the indicated primer system.

The term “calicivirus primer system” as used herein refers to a combination of two nucleic acid primers having the ability to amplify nucleic acid provided that the sequence of each nucleic acid primer is from 15 to 50 nucleotides in length and is such that it aligns without a mismatch to a sequence, or its complement, set forth in a GenBank gi number listed in Table 6. For example, each primer of a calicivirus primer system provided herein can be from 15 to 45 nucleotides the length. In some cases, each calicivirus primer of a primer system provided herein can range from 20 to 40 nucleotides (e.g., from 20 to 35 nucleotides, from 20 to 30 nucleotides, or from 21 to 28 nucleotides). The calicivirus primer systems provided herein can be selected such that the length of amplified target nucleic acid, if present within an amplification reaction, would be between 100 and 400 nucleotides (e.g., between 150 and 350 nucleotides, between 175 and 325 nucleotides, or between 200 and 300 nucleotides). The theoretical melting temperature of each primer of a calicivirus primer system provided herein can be between 58° C. and 62° C. (e.g., between 59° C. and 61° C.). Each calicivirus primer system provided herein can be used to amplify nucleic acid present in a calicivirus.

TABLE 6
Representative gi numbers for each calicivirus primer system.

| Primer | |
|---|---|
| System No. | gi number |
| 22, 23, 24 | 57470970; 37783558; and 15315610 |

The calicivirus primer systems provided herein can share unifying advantageous features. For example, each calicivirus primer system provided herein can amplify nucleic acid from caliciviruses. In addition, primer systems provided herein can be selected such that the length of amplified viral nucleic acid would be between 100 and 400 nucleotides. Moreover, the theoretical melting temperature of the calicivirus primer systems provided herein can be uniformly between 58° C. and 62° C., and the length of each primer of the calicivirus primer systems provided herein can range from 15 to 50 nucleotides (e.g., from 21 to 28 nucleotides). These unifying characteristics can contribute to the effective detection of nucleic acid from caliciviruses present within samples.

The primer systems listed in Table 5 can be used effectively to detect a large group of different caliciviruses. For example, primer system number 22 can have the ability to detect calicivirus nucleic acid sequences associated with 36 different GenBank gi numbers.

In some cases, a system or diagnostic device provided herein can contain at least one primer system set forth in Table 7 (e.g., at least two primer systems set forth in Table 7). In addition to containing any one or more of the primer systems set forth in Table 7 in any combination, a diagnostic device can contain primer systems not listed in Table 7. For example, a diagnostic device can contain a primer system similar to primer system number 25 with the exception that each nucleic acid primer is two nucleotides shorter than those of primer system number 25. In some cases, a diagnostic device can contain a primer pair designed to amplify host nucleic acid (e.g., human genomic nucleic acid or mRNA).

TABLE 7
Optimal coronavirus primer systems for coronaviruses.

| Primer | | | | | |
|---|---|---|---|---|---|
| System No. | Primer Sequence | SEQ ID NO: | Length | Tm | Hits* |
| 25 | TGGTTTTATAGGTGCCACAATTC | 37 | 23 | 60.1 | 64 |
| | GCCAATTCAGTTTGTTTACCAG | 38 | 22 | 58.7 | |
| 26 | TTTATAGGTGCCACAATTCGTCTAC | 39 | 25 | 60.6 | 63 |
| | GCCAATTCAGTTTGTTTACCAG | 38 | 22 | 58.7 | |
| 27 | TTTTATAGGTGCCACAATTCGTCTA | 40 | 25 | 61.0 | 63 |
| | GCCAATTCAGTTTGTTTACCAG | 38 | 22 | 58.7 | |

*total number of different gi numbers that is available in GenBank with nucleic acid sequences aligning with each primer of the indicated primer system.

The term “coronavirus primer system” as used herein refers to a combination of two nucleic acid primers having the ability to amplify nucleic acid provided that the sequence of each nucleic acid primer is from 15 to 50 nucleotides in length and is such that it aligns without a mismatch to a sequence, or its complement, set forth in a GenBank gi number listed in Table 8. For example, each primer of a coronavirus primer system provided herein can be from 15 to 45 nucleotides the length. In some cases, each primer of a coronavirus primer system provided herein can range from 20 to 40 nucleotides (e.g., from 20 to 35 nucleotides, from 20 to 30 nucleotides, or from 21 to 28 nucleotides). The coronavirus primer systems provided herein can be selected such that the length of amplified target nucleic acid, if present within an amplification reaction, would be between 100 and 400 nucleotides (e.g., between 150 and 350 nucleotides, between 175 and 325 nucleotides, or between 200 and 300 nucleotides). The theoretical melting temperature of each primer of a coronavirus primer system provided herein can be between 58° C. and 62° C. (e.g., between 59° C. and 61° C.). Each coronavirus primer system provided herein can be used to amplify nucleic acid present in a coronavirus.

TABLE 8
Representative gi number for each primer system.

| Primer | |
|---|---|
| System No. | gi number |
| 25, 26, 27 | 57117323 |

The coronavirus primer systems provided herein can share unifying advantageous features. For example, each coronavirus primer system provided herein can amplify nucleic acid from coronaviruses. In addition, coronavirus primer systems provided herein can be selected such that the length of amplified viral nucleic acid would be between 100 and 400 nucleotides. Moreover, the theoretical melting temperature of the coronavirus primer systems provided herein can be uniformly between 58° C. and 62° C., and the length of each primer of the coronavirus primer systems provided herein can range from 15 to 50 nucleotides (e.g., from 21 to 28 nucleotides). These unifying characteristics can contribute to the effective detection of nucleic acid from coronaviruses present within samples.

The primer systems listed in Table 7 can be used effectively to detect a large group of different coronaviruses. For example, primer system number 25 can have the ability to detect coronavirus nucleic acid sequences associated with 64 different GenBank gi numbers.

In some cases, a system or diagnostic device provided herein can contain at least one primer system set forth in Table 9 (e.g., at least two primer systems set forth in Table 9). In addition to containing any one or more of the primer systems set forth in Table 9 in any combination, a diagnostic device can contain primer systems not listed in Table 9. For example, a diagnostic device can contain a primer system similar to primer system number 28 with the exception that each nucleic acid primer is two nucleotides shorter than those of primer system number 28. In some cases, a diagnostic device can contain a primer pair designed to amplify host nucleic acid (e.g., human genomic nucleic acid or mRNA).

TABLE 9

Optimal respiratory syncytial virus primer systems
for respiratory syncytial viruses.

| Primer | | | | | |
|---|---|---|---|---|---|
| System No. | Primer Sequence | SEQ ID NO: | Length | Tm | Hits* |
| 28 | AAAAACACAACAACAACCCAAATAC | 41 | 25 | 60.3 | 330 |
| | TTGAACACTTCAAAGTGAAAATCAT | 42 | 25 | 59.1 | |
| 29 | AAAAACACAACAACAACCCAAATAC | 41 | 25 | 60.3 | 321 |
| | CAAAGTTGAACACTTCAAAGTGAAA | 43 | 25 | 59.8 | |
| 30 | AAAAACACAACAACAACCCAAATA | 44 | 24 | 59.6 | 320 |
| | CAAAGTTGAACACTTCAAAGTGAAA | 43 | 25 | 59.8 | |

*total number of different gi numbers that is available in GenBank with nucleic acid sequences aligning with each primer of the indicated primer system.

The term “respiratory syncytial virus primer system” as used herein refers to a combination of two nucleic acid primers having the ability to amplify nucleic acid provided that the sequence of each nucleic acid primer is from 15 to 50 nucleotides in length and is such that it aligns without a mismatch to a sequence, or its complement, set forth in a GenBank gi number listed in Table 10. For example, each primer of a respiratory syncytial virus primer system provided herein can be from 15 to 45 nucleotides the length. In some cases, each primer of a respiratory syncytial virus primer system provided herein can range from 20 to 40 nucleotides (e.g., from 20 to 35 nucleotides, from 20 to 30 nucleotides, or from 21 to 28 nucleotides). The primer systems provided herein can be selected such that the length of amplified target nucleic acid, if present within an amplification reaction, would be between 100 and 400 nucleotides (e.g., between 150 and 350 nucleotides, between 175 and 325 nucleotides, or between 200 and 300 nucleotides). The theoretical melting temperature of each primer of a respiratory syncytial virus primer system provided herein can be between 58° C. and 62° C. (e.g., between 59° C. and 61° C.). Each respiratory syncytial virus primer system provided herein can be used to amplify nucleic acid present in a respiratory syncytial virus.

TABLE 10
Representative gi numbers for each primer system.

| Primer | |
|---|---|
| System No. | gi number |
| 28 | 37790367; 45386490; 37790483; 485873; 21929777; |
| | 52352480; 21929881; 21929779; 52352474; 37790463; |
| | 37790583; 21929761; and 61373253 |
| 29 | 37790367; 45386490; 37790487; 485873; 21929777; |
| | 52352480; 21689580; 21929881; 21729393; 37790463; |
| | 37790583; 21929779; 21929761; and 61373253 |
| 30 | 37790367; 45386490; 37790487; 485873; 21929777; |
| | 52352480; 21929881; 37790463; 37790583; 21929779; |
| | 21929761; and 61373253 |

The respiratory syncytial virus primer systems provided herein can share unifying advantageous features. For example, each respiratory syncytial virus primer system provided herein can amplify nucleic acid from respiratory syncytial viruses. In addition, respiratory syncytial virus primer systems provided herein can be selected such that the length of amplified viral nucleic acid would be between 100 and 400 nucleotides. Moreover, the theoretical melting temperature of the respiratory syncytial virus primer systems provided herein can be uniformly between 58° C. and 62° C., and the length of each primer of the respiratory syncytial virus primer systems provided herein can range from 15 to 50 nucleotides (e.g., from 21 to 28 nucleotides). These unifying characteristics can contribute to the effective detection of nucleic acid from respiratory syncytial viruses present within samples.

The primer systems listed in Table 9 can be used effectively to detect a large group of different respiratory syncytial viruses. For example, primer system number 28 can have the ability to detect respiratory syncytial virus nucleic acid sequences associated with 330 different GenBank gi numbers.

In some cases, a system or diagnostic device provided herein can contain at least one primer system set forth in Table 11 (e.g., at least two primer systems set forth in Table 11). In addition to containing any one or more of the primer systems set forth in

Table 11 in any combination, a diagnostic device can contain primer systems not listed in Table 11. For example, a diagnostic device can contain a primer system similar to primer system number 31 with the exception that each nucleic acid primer is two nucleotides shorter than those of primer system number 31. In some cases, a diagnostic device can contain a primer pair designed to amplify host nucleic acid (e.g., human genomic nucleic acid or mRNA).

TABLE 11
Optimal SARS coronavirus primer systems for SARS coronaviruses.

| Primer | | | | | |
|---|---|---|---|---|---|
| System No. | Primer Sequence | SEQ ID NO: | Length | Tm | Hits* |
| 31 | ATGTAGTTCGTGATCTACCTTCTGG | 45 | 25 | 60.0 | 244 |
| | TTTAAATAGCCAACAAAATAGGCTG | 46 | 25 | 59.9 | |
| 32 | ATGTAGTTCGTGATCTACCTTCTGG | 45 | 25 | 60.0 | 244 |
| | AAGGCTGTAAGAATGGCTCTPAAAT | 47 | 25 | 60.1 | |
| 33 | GTTCGTGATCTACCTTCTGGTTTTA | 48 | 25 | 60.0 | 244 |
| | TTTAAATAGCCAACAAAATAGGCTG | 46 | 25 | 59.9 | |

*total number of different gi numbers that is available in GenBank with nucleic acid sequences aligning with each primer of the indicated primer system.

The term “SARS coronavirus primer system” as used herein refers to a combination of two nucleic acid primers having the ability to amplify nucleic acid provided that the sequence of each nucleic acid primer is from 15 to 50 nucleotides in length and is such that it aligns without a mismatch to a sequence, or its complement, set forth in a GenBank gi number listed in Table 12. For example, each primer of a SARS coronavirus primer system provided herein can be from 15 to 45 nucleotides the length.

In some cases, each primer of a SARS coronavirus primer system provided herein can range from 20 to 40 nucleotides (e.g., from 20 to 35 nucleotides, from 20 to 30 nucleotides, or from 21 to 28 nucleotides). The SARS coronavirus primer systems provided herein can be selected such that the length of amplified target nucleic acid, if present within an amplification reaction, would be between 100 and 400 nucleotides (e.g., between 150 and 350 nucleotides, between 175 and 325 nucleotides, or between 200 and 300 nucleotides). The theoretical melting temperature of each primer of a SARS coronavirus primer system provided herein can be between 58° C. and 62° C. (e.g., between 59° C. and 61° C.). Each SARS coronavirus primer system provided herein can be used to amplify nucleic acid present in a SARS coronavirus.

TABLE 12
Representative gi numbers for each primer system.

| Primer | |
|---|---|
| System No. | gi number |
| 31, 32, 33 | 37624337; 32187341; 42741238; 34482144; 44894074; |
| | 34482141; 34482138; 37624327; 49036420; 37624324; |
| | 42563750; 37624323; 37624331; 37624325; 37624347; |
| | 37624320; 39980888; 45384966; 32454341; 45384973; |
| | 53759059; 92429688; and 41352883 |

The SARS coronavirus primer systems provided herein can share unifying advantageous features. For example, each SARS coronavirus primer system provided herein can amplify nucleic acid from SARS coronaviruses. In addition, SARS coronavirus primer systems provided herein can be selected such that the length of amplified viral nucleic acid would be between 100 and 400 nucleotides. Moreover, the theoretical melting temperature of the SARS coronavirus primer systems provided herein can be uniformly between 58° C. and 62° C., and the length of each primer of the SARS coronavirus primer systems provided herein can range from 15 to 50 nucleotides (e.g., from 21 to 28 nucleotides). These unifying characteristics can contribute to the effective detection of nucleic acid from SARS coronaviruses present within samples.

The primer systems listed in Table 11 can be used effectively to detect a large group of different SARS coronaviruses. For example, primer system number 31 can have the ability to detect SARS coronavirus nucleic acid sequences associated with 244 different GenBank gi numbers.

Any method can be used to make the primers of a primer system provided herein. For example, chemical synthesis techniques such as those described elsewhere (Beaucage and Caruthers, Tetrahedron Lett., 22:1859-62 (1981)) can be used. In addition, nucleic acid primers can be obtained from commercial vendors such as MWG Biotech, Invitrogen, and Operon.

Any method can be use to make a system or diagnostic device provided herein. For example, a diagnostic device provided herein can be made as follows. A 384-well master plate containing 125 μL of one or more primer systems in dioinized water at a working concentration of 100 nmole/1 μL of each primer can be constructed. The master plate can be used as a template source, and 1 μL of each master plate well can be transferred to corresponding wells on a 384-well microfluidic card. Spotted reagents can be allowed to dry at room temperature before the final plastic laminate layer of the microfluidic card is attached.

The primer systems provided herein can be used separately or in combinations. Such combinations can contain 2, 3, 4, 5, 10, 15, or more different primer systems listed in Table 1, 3, 5, 7, 9, or 11. When making a combination, any two or more of the provided primer systems can be arranged into any combination. For example, the first primer system listed in Table 1 for each of the targets can be used to make a collection of six different primer systems. Other combinations include, without limitation, a combination of six different primer systems containing the second primer system listed in Table 1 for each of the targets; a combination of six different primer systems containing the third primer system listed in Table 1 for each of the targets; and a combination of 18 different primer systems containing the first, second, and third primer system listed in Table 1 for each of the targets.

The diagnostic devices and primer systems provided herein can be used to detect viruses (e.g., influenza A viruses, adenoviruses, caliciviruses, coronaviruses, respiratory syncytial viruses, or SARS coronaviruses) present within samples. For example, a sample can be obtained from a human (or other animal such as a bird) and used in an amplification reaction to determine whether or not a viral nucleic acid (e.g., an influenza A virus' nucleic acid, an adenovirus' nucleic acid, a calicivirus' nucleic acid, a coronavirus' nucleic acid, a respiratory syncytial virus' nucleic acid, or a SARS coronavirus' nucleic acid) is present in the sample. Any type of sample can be used including, without limitation, a biopsy (e.g., punch biopsy, aspiration biopsy, excision biopsy, needle biopsy, or shave biopsy), a tissue section, lymph fluid, mucus, blood, serum, and saliva samples. A sample can be obtained from a human or any other animal (e.g., birds, pigs, and horses) suspected to contain a virus (e.g., an influenza A virus, an adenovirus, a calicivirus, a coronavirus, a respiratory syncytial virus, or a SARS coronavirus). In some cases, a sample can be obtained from a mammal (e.g., a human) using a swab (e.g., an OmniSwab; Whatman). The presence of an amplification product following an amplification reaction using, for example, a human's mucus sample and a primer system provided herein can indicate that that sample contains a virus (e.g., an influenza A virus, an adenovirus, a calicivirus, a coronavirus, a respiratory syncytial virus, or a SARS coronavirus). In such a case, the human can be diagnosed as being infected with the virus.

Some sample types can be pre-processed to enhance sample quality. For example, a mucus sample can be treated with a mucolytic agent to liquefy mucus within a mucus sample. Samples can be processed to concentrate the nucleic acid and render it in a form to facilitate successful PCR reactions. This includes, but is not limited to, common methods to disrupt bilipid membranes, such as the use of detergents, digestion of protein complexes, such as the use of proteinase K, and reduction of polymerase inhibitors through the use of selective affinity columns. Commercial kits for purification of DNA, RNA, or total nucleic acid are readily available from, for example, Qiagen and Roche. In some cases, a sample can be processed using a Qiagen QIAmp Viral RNA Mini Kit.

Any type of amplification reaction can be used in conjunction with the primer systems provided herein including, without limitation, those set forth in Table 1, 3, 5, 7, 9, or 11. For example, common PCR reactions designed to amplify nucleic acid from DNA or RNA can be used. Detection of RNA viruses can be accomplished by synthesizing cDNA from RNA sequence templates. cDNA synthesis can be accomplished using standard methods using, for example, RNA-dependant DNA polymerases, such as reverse transcriptase. Such reactions can be primed with random oligonucleotide sequences, such as random hexamers and octamers, or by sequence specific oligonucleotide primers, including the same primers used for the PCR reaction. The cDNA synthesis can be performed in a separate reaction vessel from the subsequent PCR reaction (commonly referred to as two-step rtPCR) or in the same reaction vessel as the PCR reaction (commonly referred to as single-step rtPCR).

Purified DNA and cDNA samples can be pooled and added to a PCR master mix containing water, salt buffers, magnesium ions, nucleotide monomers (dATP, dCTP, dGTP and dTTP), native or engineered Thermus aquaticus DNA-dependant DNA polymerase, and an intercalating dye, such as Sybr Green or LC Green. The master mix and sample can then be added to a single loading port of a microfluidic card and dispersed to all the reaction wells using centrifugation. The cards can then be scored to isolate and seal each reaction chamber prior to thermocycling. The cards can be individually thermocycled using commodity block thermocyclers or many cards thermocycled simultaneously using air- or water-based thermocyclers such as the BioOven or the H2OBIT, respectively.

Positive PCR amplification reactions can be detected during thermocycling for quantitative or qualitative analysis (real time PCR) or after completion of thermocycling (qualitative end-point PCR). Signals can be detected through fluorescence-channel emission of substrate bound intercalating dyes using commodity real-time PCR capable PCR platforms or by end-point reads using microplate scanner platforms. Both types of platforms can be used for melting-point analysis for validation of positive signals.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A device comprising a housing having a plurality of locations, wherein each of said locations contains an influenza virus primer system, wherein the primers of each influenza virus primer system are between 18 and 28 nucleotides in length and have a theoretical melting temperature between 58° C. and 62° C., wherein said device comprises at least one influenza virus primer system capable of producing an amplification product diagnostic for an H1 target, at least one influenza virus primer system capable of producing an amplification product diagnostic for an H2 target, at least one influenza virus primer system capable of producing an amplification product diagnostic for an H3 target, at least one influenza virus primer system capable of producing an amplification product diagnostic for an H5 target, at least one influenza virus primer system capable of producing an amplification product diagnostic for an N1 target, and at least one influenza virus primer system capable of producing an amplification product diagnostic for an N2 target, and wherein each amplification product, when produced, is between 100 and 400 nucleotides in length.

2. The device of claim 1, wherein each of said locations is a chamber.

3. The device of claim 1, wherein each of said locations is a well.

4. The device of claim 1, wherein two or more of said locations comprise two or more influenza virus primer systems.

5. The device of claim 4, wherein each influenza virus primer system of said two or more primer systems of a location is capable of producing an amplification product diagnostic for a different target of influenza A viruses.

6. The device of claim 1, wherein the primers of each influenza virus primer system are between 23 and 27 nucleotides in length.

7. The device of claim 1, wherein the primers of each influenza virus primer system have a theoretical melting temperature between 59° C. and 61° C.

8. The device of claim 1, wherein said housing comprises additional locations, wherein each of said additional locations contains a primer pair.

9. The device of claim 8, wherein at least one of said additional locations comprises a primer pair capable of producing an amplification product from human nucleic acid.

10. The device of claim 1, wherein each of said locations comprises an intercalating dye, and wherein each amplification product, when produced, is labeled with said intercalating dye.

11. The device of claim 10, wherein said intercalating dye is a green fluorescent dye.

12. The device of claim 10, wherein said intercalating dye is SYBR Green, LC Green, or SYTO9.

13. The device of claim 1, wherein each amplification product, when produced, is between 100 and 300 nucleotides in length.

14. A method for detecting an influenza A virus within a sample, wherein said method comprises:
(a) performing a nucleic acid amplification reaction using said sample as a source of template and a diagnostic device, wherein said device comprises a housing having a plurality of locations, wherein each of said locations contains an influenza virus primer system, wherein the primers of each influenza virus primer system are between 18 and 28 nucleotides in length and have a theoretical melting temperature between 58° C. and 62° C., wherein said device is capable of producing an amplification product diagnostic for at least six different targets of influenza A viruses, and wherein each amplification product, when produced, is between 100 and 400 nucleotides in length, and
(b) determining which locations of said device contain a primer system that resulted in the formation of amplification product, thereby detecting an influenza A virus.

15. The method of claim 14, wherein said sample is a mucus sample from a human.

16. The method of claim 14, wherein said determining step (b) is performed in using a dye reader device configured to receive said diagnostic device.

17. The method of claim 14, wherein each of said locations comprises an intercalating dye, wherein each amplification product, when produced, is labeled with said intercalating dye, and wherein determining which locations of said device contain a primer system that resulted in the formation of amplification product is based on a signal from said dye.

18. The method of claim 14, wherein said amplification reaction and said determining step (b) are performed in a machine configured to receive said diagnostic device, said machine comprising a thermal cycler device and a dye reader device.

19. The method of claim 18, wherein said machine is capable of providing output indicating the presence of said influenza A virus.

20. The method of claim 18, wherein said machine is capable of providing output indicating the primer systems that detected the presence of said influenza A virus.

21-120. (canceled)
